(19) United States
(12) Patent Application Publication
(10) Pub. No.: US 2024/0350305 A1
(43) Pub. Date: Oct. 24, 2024

(54) DISPENSER FOR DRIP-DISPENSING A STERILE LIQUID PRODUCT CONTAINING A SURFACTANT

(71) Applicant: LABORATOIRES THEA, CLERMONT-FERRAND (FR)

(72) Inventor: Benjamin QUAGLIA, Châteaugay (FR)

(21) Appl. No.: 18/713,721

(22) PCT Filed: Nov. 23, 2022
(86) PCT No.: PCT/FR2022/052164

§ 371 Date: May 28, 2024

(30) Foreign Application Priority Data
Nov. 29, 2021 (FR) 2112639

(51) Int. Cl. A61F 9/00

(52) U.S. Cl. CPC A61F 9/0008

(57) ABSTRACT

A dispenser for drip-dispensing a sterile liquid product containing a surfactant, including a reservoir having a wall deforms when pressure is applied and spontaneously returns to its original shape upon release. The reservoir includes a flexible bag in the internal space for holding the liquid product, an air inlet for letting air in between the wall of the reservoir and the flexible bag, and a head that comprises a drip end piece comprising a dispensing opening. The dispenser also includes a selectively permeable, hydrophilic sterilizing microporous membrane arranged so that the liquid from the flexible bag passes therethrough on the way to being dispensed and a device that is configured to keep the membrane wetted with the liquid product from the reservoir between two instances of the liquid product being dispensed.

The present invention relates to a dispenser for drip-dispensing a sterile liquid product containing a surfactant. The dispenser proposed according to the present invention has a particular advantage for packaging a preservative-free liquid ophthalmic product, containing a surfactant.

There are liquid products that have to be drip-dispensed in many fields in industry, in particular in the pharmaceutical field, the cosmetic field, the food industry, etc. These products are used in small doses, of one or a few drips coming from a dispenser during each use, whereas it is necessary to keep the rest of the product contained in the dispenser for a certain length of time, generally several days or even several weeks. The product contained in the dispenser must generally be kept away from any contamination by bacterial agents or others coming from outside the dispenser.

This is in particular the case with pharmaceutical compositions, including in particular ophthalmic solutions, which constitute the preferential application of the present invention. There is furthermore a particular interest in proposing preservative-free ophthalmic solutions. This is because the products used as preservative in ophthalmic solutions, for ensuring sterility of the ophthalmic products with respect to bacteria and fungi, may have significant undesirable effects.

Preservatives, in particular benzalkonium chloride, are irritant for the eyes and may impair the lacrimal film, and may cause tissue impairment by apoptosis. The long-term use of preservatives has undesirable consequences on the eye.

Thus, the use of preservative-free ophthalmic products is very often recommendable. A first solution for preserving the sterility of such preservative-free ophthalmic products consists in packaging them in single doses, also referred to as “unidoses”. Packaging in a unidose is however not applicable to all products, and gives rise to a great deal of waste, the packaging of each dose having to be discarded after use. For treatment of long duration, packaging in a unidose does therefore not appear to be appropriate. In this context, dispensers containing several doses, or “multidose” dispenser, configured to ensure sterility of the product that they contain, including after opening and delivery of the first dose, have been developed. The aim of these dispensers is to preserve the sterility in the dispenser as long as the consumption of the product lasts until the content of the dispenser is completely exhausted.

For example, the document FR2816600 discloses a dispenser of the drip type, the reservoir of which includes a deformable wall making it possible to create overpressure causing the product contained to pass through a sterilising membrane. The surface of this membrane is partially hydrophilic and partially hydrophobic, and more particularly selectively permeable to aqueous liquids in its hydrophilic part and selectively permeable to air in its hydrophobic part. Thus the liquid passes through the membrane in its hydrophilic part during dispensing before reaching a drip-dispensing end piece, while air can be sucked into the dispenser through the hydrophobic part to compensate for the volume of liquid dispensed when the reservoir is no longer compressed and returns to its initial shape. A porous pad allows regulation of the flow in the dispenser, necessary for proper formation of drips.

This type of dispenser is completely satisfactory for packaging and dispensing many preservative-free sterile products, and in particular preservative-free liquid ophthalmic products.

Nevertheless, they are not adapted to dispensing some products. In particular, ophthalmic products containing a surfactant cannot be dispensed satisfactorily by this type of dispenser. This is because surfactants interact with the hydrophobic part of the membrane (the latter being obtained by a localised physicochemical treatment of the hydrophobic membrane) and make it hydrophilic. This prevents air returning to the dispenser, necessary for compensating for the quantity of liquid dispensed. The consequence of this is an increase in the negative pressure in the dispenser as it is used, which can cause residual deformation of the dispenser and/or rupture of the membrane.

A surfactant (or surfactant agent or surface agent) is a compound that modifies the surface tension between two surfaces. These are compounds that have two parts with different polarities, respectively lipophilic and hydrophilic. They make it possible to solubilise two immiscible phases. In the context of ophthalmic products, surfactants are thus used as a solubilisation agent. The terms surfactant and solubilisation agent are thus equivalent in the present document.

The document FR2955842 discloses an improved dispenser reducing the formation of foam as well as the retention of air in the porous pad. Nevertheless, though the dispenser proposed substantially improves the quality of the dispensing of a product containing either an active principle itself having surfactant properties, or surfactant additives used as solubilising agents, or other excipients, such as certain viscosing or lubricating agents in the family of polyvinyl derivatives or the family of polyethylene glycols, it does not solve the problem of the incompatibility of certain surfactants with respect to the hydrophobic part of the membrane used.

The document FR2770495 discloses a dispenser for a preservative-free liquid ophthalmic product that would be adapted to the dispensing of a product containing a surfactant. Nevertheless, the dispenser proposed is based on a reservoir with a flexible wall, manually deformable as its internal volume gradually decreases towards a rigid neck, i.e. in practice on a reservoir deformable in an accordion, the manipulation of which is not perfectly easy for the user.

The document EP1985543 presents a dispenser for an ophthalmic product. The reservoir of the dispenser is of the “delamination” type, which means that the internal layer of the reservoir can separate from the rest of the walls to form a deformable internal bag. This dispenser includes a nonreturn valve through which the product emerging from the dispenser passes and can include, downstream of this valve, a filter for filtering certain bacteria. This dispenser however requires the use of a very effective nonreturn valve, and has a complex configuration when a filtration function is implemented.

The invention aims to propose a dispenser for a sterile liquid product containing at least one surfactant that solves at least some of the problems mentioned above.

Thus the invention relates to a dispenser for drip-dispensing a sterile liquid product containing a surfactant, including a reservoir including a wall defining an internal volume and adapted to deform under the effect of a pressure exerted on the reservoir by a user of the dispenser and to spontaneously resume its original shape after said pressure is released. The reservoir furthermore includes a flexible bag in the internal volume defined by the wall and adapted to contain the sterile liquid product. The dispenser includes an air-inlet means between the wall of the reservoir and the flexible bag, and a head attached to a neck of the reservoir and including a drip end piece including a dispensing opening. The dispenser furthermore includes a hydrophilic sterilising microporous membrane, disposed so as to have the liquid coming from the flexible bag passing through it with a view to delivery of said liquid through the drip end piece.

The dispenser includes a device configured to keep the membrane wetted by liquid product coming from the reservoir between two dispensings of liquid product. The microporous membrane is selectively permeable to liquid, i.e. it enables an aqueous liquid to pass while it opposes the passage of gases when it is wetted, so that the membrane opposes the return of air into the flexible bag of the reservoir when the dispenser resumes its original shape after a dispensing of the sterile liquid product, the volume of liquid dispensed being compensated for by the entry of a corresponding volume of air between the wall of the reservoir and the flexible bag via the air-inlet means.

The present invention thus proposes a drip-dispensing dispenser perfectly adapted to products containing a solubilisation agent (surfactant). The dispenser proposed is simple to use for the user, in that it enables product to be dispensed by simple pressure on the reservoir, which is intuitive. Furthermore, the flexible bag that is present in the internal volume defined by the wall of the reservoir allows complete dispensing of the product that it contains, i.e. all the doses of product contained in the flexible bag can actually be dispensed. Products can also be dispensed while the dispenser is positioned in various orientations. This is because, as doses of product are dispensed, the volume of product dispensed is compensated for by the entry of an equivalent volume of air between the deformable wall of the reservoir and the flexible bag. The volume of the flexible bag adapts to that of the product that it contains. The product contained in the flexible bag thus remains in the immediate vicinity of the dispensing head, and no air bubble forms in the flexible bag. When the user applies a pressure to the deformable wall, the air present between the deformable wall of the reservoir and the flexible bag is prevented from emerging from the space (whether by a means that automatically prevents this emergence of the air or through an action of the user), so that the flexible bag is itself pressurised, causing the expulsion of the product through the hydrophilic membrane and the drip end piece.

The sterilising membrane guarantees sterility of the product during delivery thereof. It also protects the product contained in the reservoir against any contamination coming from outside the dispenser. The dispenser proposed in the invention also benefits from the character of the sterilising membrane of being selectively permeable to water (or more generally to aqueous liquids) to avoid return of air into the flexible bag after the product is dispensed. In other words, the membrane, the filtration capability of which guarantees the sterility of the product contained in the dispenser, does not allow air to return into the dispenser and thus serves as a nonreturn device for the dispenser.

In order to guarantee the total absence of the passage of air, the membrane must be wetted. This is necessarily the case immediately after the product is dispensed, so that the membrane correctly fulfils its role of nonreturn device and prevents air being sucked into the flexible bag in compensation for the volume of liquid product dispensed. Nevertheless, to ensure that the membrane remains wetted between two dispensings of product, which are generally spaced apart by several hours if the product is an ophthalmic product, it is proposed to provide a device adapted to guarantee that the membrane remains wetted. This device can be formed in various ways. A reserve of liquid can be formed upstream of the membrane, or a very small quantity of liquid can be maintained downstream of the membrane.

Throughout the present document the terms “upstream” and “downstream” are meant considering the direction of flow of the liquid product when it is delivered, i.e. from the reservoir until it emerges from the end piece of the dispenser.

The dispenser proposed in the invention can have a simple general configuration. In particular, it is not necessary to provide a perfectly airtight nonreturn device in addition to the membrane to prevent the return of air into the flexible bag of the reservoir when the wall resumes its original shape after having been deformed by pressure to cause dispensing of a few drips of product.

The flexible bag can be formed by delamination of an internal layer of the reservoir. As an alternative to a flexible bag present as from manufacture in the reservoir of the dispenser, a flexible bag obtained by delamination of the wall of the reservoir is a simple solution, industrially mastered, and inexpensive, to obtain the function sought in the invention.

Alternatively, the flexible bag can be a retractable flexible bag that is attached. The use of such a retractable flexible bag makes it possible in particular to select a bag having a nominal capacity corresponding precisely to the initial volume of liquid product to be packaged, while using one and the same deformable wall for various volumes of product to be packaged.

The air-inlet means can be formed by a hole in the wall of the reservoir adapted to be closed off by a finger of the user.

This is a particularly simple solution for preventing the emergence of the air contained between the wall of the reservoir and the flexible bag when product is dispensed towards the outside of the reservoir, i.e. when pressure is applied to the reservoir. An adapted positioning of the hole provided in the wall makes use of the dispenser perfectly intuitive. The hole can thus advantageously be positioned between a zone of the reservoir where a finger of the user naturally comes to be placed when they hold the dispenser with a view to dispensing the product contained by the dispenser. The hole can thus be positioned on a lateral wall of the reservoir, in particular in proximity to the neck of the reservoir, or alternatively on the bottom of the reservoir.

Alternatively, the air-inlet means can include a unidirectional valve, configured to allow air to enter between the wall of the reservoir and the flexible bag and to prevent the air present between the wall of the reservoir and the flexible bag from emerging towards the outside of the reservoir.

According to this configuration, the emergence of air towards the outside of the reservoir is automatically prevented, which makes use of the reservoir extremely simple, since the user has no particular action to perform (however simple) compared with a conventional dispenser with deformable reservoir.

The device configured to keep the membrane wetted between two instillations can be a removable cap, attached to the dispensing head.

A cap constitutes a simple and effective way of providing impermeability downstream (on the same side as the drip end piece) of the membrane. Such impermeability prevents the return of the liquid that wets the membrane towards the flexible bag between two instillations.

The device configured to keep the membrane wetted between two instillations can include a valve positioned between the membrane and the dispensing opening of the drip end piece.

It is notable that this valve does not aim to fulfil the function of anti-resuction of air into the reservoir, which is guaranteed in the invention by the hydrophilic membrane, selectively permeable to water. Such a valve simply ensures the maintenance of the liquid product wetting the membrane at the latter, in particular between two successive dispensings of products. A simple inexpensive valve can thus be used. The valve can be a ball valve, a valve with deformable lamellae, etc.

The dispenser can include a porous pad located between the reservoir and the microporous membrane and disposed so as to have the liquid coming from the flexible bag passing through it with a view to delivery of said liquid through the drip end piece. The porous pad upstream of the membrane can avoid the liquid product coming into contact with the membrane before the first dispensing of product. Before first use, the liquid thus remains perfectly confined in the reservoir, and no drop of liquid risks passing through the membrane and thus being located in a zone, at the end piece, where it is more difficult to guarantee sterility thereof.

The porous pad also makes it possible to create a pressure drop as the product passes towards the dispensing orifice, conjointly with the microporous membrane, which makes it possible to regulate the flow of liquid dispensed by the dispenser. In other words, the pad enables the user to more easily apportion the number of drops required, by regulating the flow of liquid emerging from the dispenser.

If the porous pad is positioned in contact with the membrane, it can also constitute a device for keeping the membrane wetted with the liquid product coming from the reservoir between two dispensings of liquid product. This arrangement must nevertheless be supplemented by a means preventing the evaporation of liquid product, for example a removable cap.

A microporous pad giving satisfaction is advantageously produced from a material that is inert with respect to the liquid contained in the dispenser. Suitable materials will in particular be high-porosity felts or foams with open pores as can be obtained from various resins of organic polymers. In the main applications of the invention, it is advantageous to produce the microporous pad in the form of a felt capsule of polyester or modified polyester resins, such as in particular low-density polyethylene resins or polyethersulfone resins.

Resins of this type or equivalent have the advantage, in the context of the invention, of lending themselves to providing a cylindrical pad, in diameters from 0.5 to 3 cm and lengths of between 0.2 and 1 cm, which has sufficient flexibility to forcibly fit imperviously in the body, advantageously cylindrical, of the dispensing head, and which, in the longitudinal direction, offers for passage of the liquid microchannels with a mean pore diameter that can be selected at between 0.3 and 10 microns.

The microporous membrane can for example have a pore size of less than 0.45 μm and preferably less than or equal to 0.22 μm.

Such a membrane offers a so-called sterilising filtration in that it prevents bacteria, fungi and, depending on the porosity chosen, certain viruses from passing. The membrane can consist of various materials, for example of the PES, nylon or PVDF type. It avoids the passage of possible contaminants during dispensing of the product (although normally the product is packaged in a sterile manner, so that no contaminant is normally present therein), but also guarantees sterility of the product contained in the reservoir by preventing the passage into the reservoir of contaminants (bacteria, or even viruses) from the exterior of the dispenser. No preservative is thus necessary in the product.

The surfaces of the dispenser located between the microporous membrane and the dispensing opening can carry a bactericidal coating, for example based on silver ions or zinc ions. The microporous membrane can include a bactericidal agent, for example based on silver ions or zinc ions.

The treatment of the surfaces of the dispenser that are downstream of the membrane and are liable to be in contact with the product during dispensing, or even in contact with product remaining in the drip end piece following dispensing, guarantees sterility of the product until it emerges from the dispenser. Such treatment generally avoids any bacterial proliferation on the wetted surfaces of the dispenser. The treatment of the surfaces can consist of the application of a coating to these surfaces. Alternatively, the material forming these surfaces can be loaded in its mass with a bactericidal agent. Bactericidal agents based on metal ions, for example silver or zinc ions, are preferred for the applications of the invention to the packaging of an ophthalmic product.

The microporous membrane is thus advantageously located as close as possible to the dispensing opening of the dispenser.

The dispenser can include in its reservoir a preservative-free sterile liquid ophthalmic product containing at least one surfactant.

This is the preferential application of the invention. The invention allows packaging of such a product in multidose form, in a simple inexpensive dispenser that is practical for the user.

The sterile liquid ophthalmic product can for example include between 0.1% and 5% surfactant by volume

The at least one surfactant can include one or more of the agents selected from:
Polyethylene glycol 400 (PEG 400);
Polyoxyethylene sorbitan monooleate (polysorbate 80);
Polyoxyethylene sorbitan monolaurate (polysorbate 20);
Ethylene oxide, propylene oxide;
Methyloxirane polymer;
Castor oil PEG-35;
Hydrogenated castor oil PEG-40;
Macrogol cetostearyl ether;
Polyoxypropyl glycol 407;
Vitamin E (polyethylene glycol succinate);
Macrogol hydroxystearate 15;
Caprylocaproyl macrogol-8 glycerides;
2-(2-ethoxyethoxy) ethanol;
Polyvinylpyrrolidone.

The liquid ophthalmic product can include at least one additive selected from the group of isotonic agents of the non-ionic type, antioxidants, buffer systems, lubricating polymers or oligosaccharides. The ophthalmic product can furthermore include one or more active agents in therapeutically effective quantity selected from the group of anti-glaucoma agents, anti-inflammatoires, immunosuppressants, antivirals, antibacterial agents or antifungal agents.

Other specific features and advantages of the invention will also become apparent in the following description.

In the appended drawings, given by way of non-limiting examples:

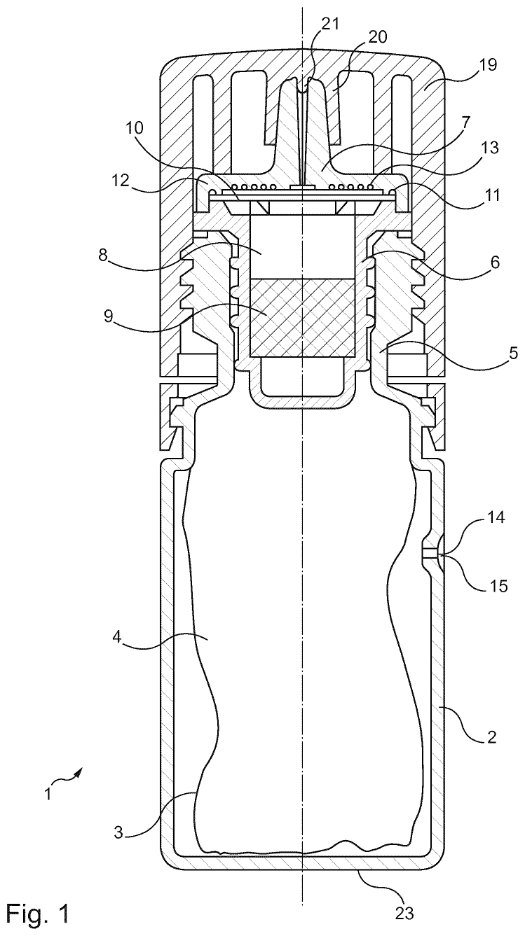

FIG. 1 shows, in a view in cross section, a dispenser according to one embodiment of the invention;

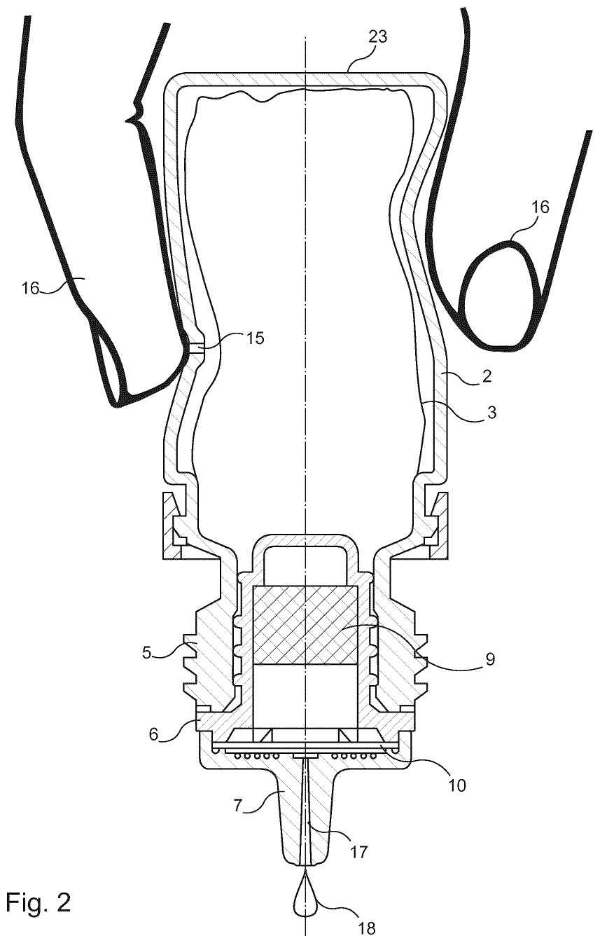

FIG. 2 shows, in a view in cross section, the dispenser of FIG. 1 when the product that it contains is being delivered;

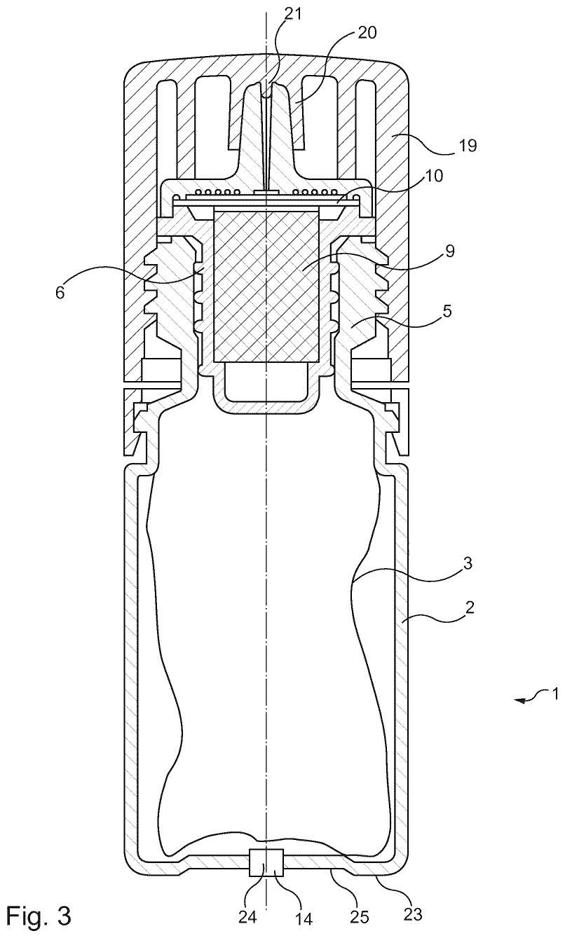

FIG. 3 shows, in a view in cross section, a dispenser according to one embodiment of the invention, illustrating several aspects that can be implemented in such a dispenser;

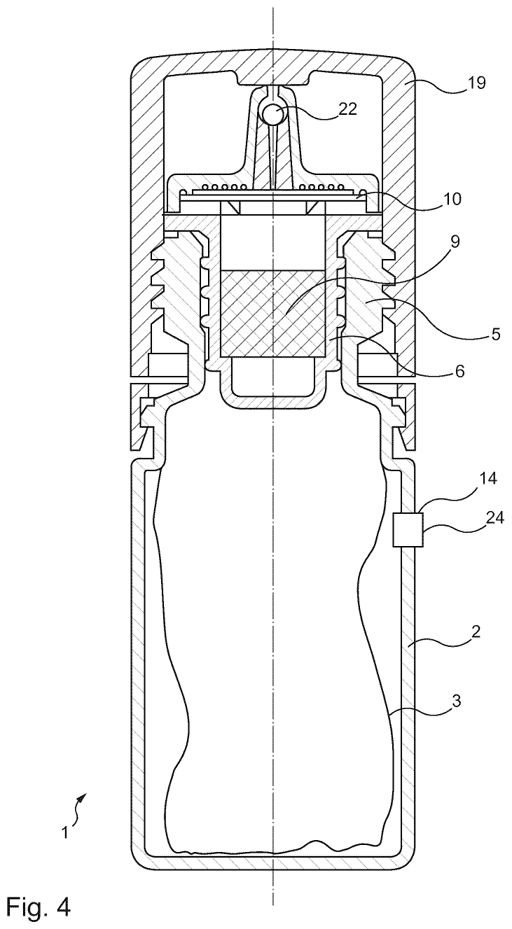

FIG. 4 shows, in a view in cross section, a dispenser according to another embodiment of the invention, illustrating several aspects that can be implemented in such a dispenser;

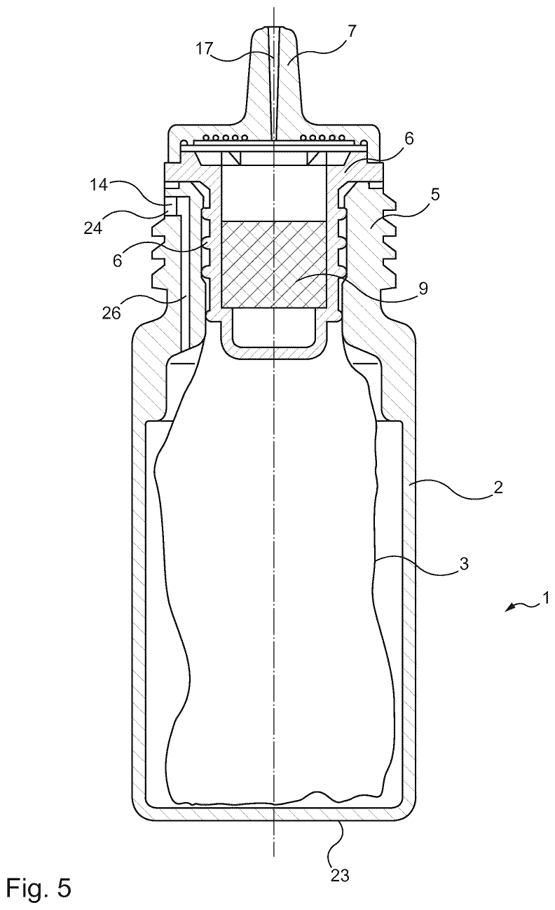

FIG. 5 shows, in a view in cross section, a dispenser according to another embodiment of the invention;

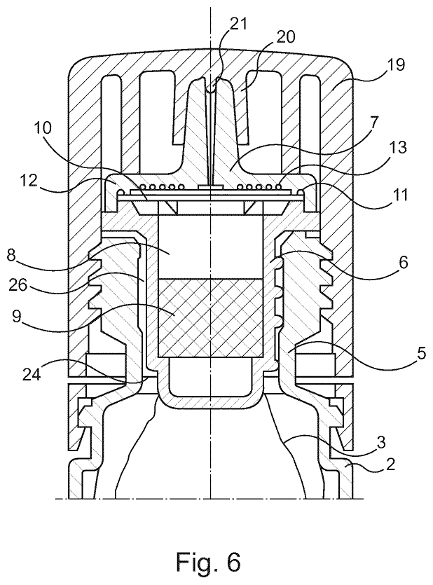

FIG. 6 shows, in a view in partial cross section, a dispenser according to yet another embodiment of the invention.

FIG. 1 shows a dispenser according to a first embodiment of the invention. The dispenser includes a reservoir 1 adapted to contain a liquid product, in particular a sterile liquid product. The reservoir 1 includes a deformable wall 2 that forms an internal volume of the reservoir 1. The wall 2 is in particular configured to be able to be deformed under a pressure exerted by the hand of a user when the latter wishes to proceed with delivering one or more drips of product.

In the example shown, the wall 2 is a cylindrical peripheral wall. The wall 2 is elastically deformable, i.e. it tends spontaneously to resume its initial shape after a pressure has ceased to be exerted thereon.

The reservoir includes, in its internal volume, a flexible bag 3. The flexible bag 3 can be an attached bag, formed from a flexible plastics material, from silicone, etc. Alternatively, the flexible bag 3 is formed by delamination of the internal surface of the wall 2, i.e. the flexible bag 3 detaches from the wall 2 during the first deliveries of product. The flexible bag forms a reception volume 4 wherein the liquid product is contained. The flexible bag must therefore have sufficient permeability characteristics to ensure good preservation of the liquid product that it contains over a long period, typically of several weeks or several months. In the example shown, the flexible bag is kept in contact with the wall 2 of the reservoir at least in proximity to a neck 5 of the reservoir.

A dispensing head is attached to the dispenser. In the example embodiment shown here, the head for dispensing liquid in drips comprises an insert 6, disposed inside the neck 5 of the dispenser. An end piece 7 (or nozzle) for drip dispensing is secured to (or formed by) the insert 6. The insert 6 is secured rigidly and sealingly in the neck 5. The insert 6 can for example be inserted forcibly in the neck 5.

The insert 6 is said to be hollowed out, in that it provides a space 8 in which a porous pad 9 is received, optional in the context of the present invention. The porous pad 9 is here cylindrical in shape matching that of the space 8. It is produced from a hydrophobic material. It can in particular be formed from a felt with a polyethylene filling. The pad 9 can for example have an equivalent porosity of approximately 100 μm. It has the effect of regulating the flow of liquid dispensed and preventing the liquid product from passing from the reservoir 1 to the end piece 7 in the absence of compression of the wall 2 of the dispenser.

The dispenser also comprises a microporous membrane 10. The microporous membrane is said to be sterilising or antibacterial in that it offers a sufficiently fine filtration to prevent the passage of all or some of the bacteria as well as, where applicable, certain viruses.

The microporous membrane is disposed downstream of the porous pad 9 and upstream of the end piece 7. The membrane protects by filtration the sterile liquid product contained in the flexible bag 3 of the reservoir 1 from external contaminations, in particular by bacteria. It also filters the liquid product contained during each dispensing.

The microporous membrane 10 has a pore size of less than 0.45 μm and preferably less than or equal to 0.22 μm, for example between 0.1 μm and 0.2 μm. It is advantageously formed from polyethersulfone. The microporous membrane used is hydrophilic (over the whole of its surface) so that it selectively allows passage of an aqueous liquid. On the other hand, it opposes the passage of gases, in particular air. This is particularly the case when it is soaked with liquid.

Thus the membrane 10 allows an aqueous liquid to pass through it under the effect of a pressure differential between its two faces, while opposing the passage of air. In particular, as long as the membrane is wetted with liquid, it strongly opposes the passage of air despite any presence of a pressure differential between the faces of the membrane.

In the example shown, this membrane 10 is interposed between the end piece 7 and the insert 6. More particularly, the membrane 10 is supported against a base 12 of the dispensing end piece 7. In the form of a disc, it is secured on its periphery by thermal welding between a peripheral ring 11 of this base and a cooperating surface present towards one end of the insert 6.

Here, the base 12 of the end piece 7 is in the form of a hollowed-out disc that fits on the insert 6. The base 12 can include, on its internal face (which is directed towards the internal volume of the reservoir 1), microchannels 13 that facilitate the draining of the liquid towards the expulsion opening.

The dispenser furthermore includes an air-inlet means 14. The air-inlet means 14 enables air to pass through the wall 2 of the dispenser 1 so as to fit between said wall 2 and the flexible bag 3. In the example shown here, the air-inlet means 14 is formed by a hole 15, for example a round hole a few millimetres in diameter. The hole 15 can be formed in a slight hollow in the wall 2.

FIG. 2 shows the dispenser of FIG. 1 when the product that it contains is being delivered. To cause the delivery of one or more drips of liquid product, a user presses the dispenser, for example between two fingers 16, thus deforming the wall 2 and reducing its internal volume. When the dispenser is manually compressed, one of the fingers 16 is placed so as to close off the hole 15, which prevents the air present between the wall 2 and the flexible bag 3 from being discharged. The result of this is an increase in the pressure in the reservoir, both with regard to the air present between the wall 2 and the flexible bag 3 and with regard to the liquid product, the pressure being transmitted by the flexible bag 3.

The pressure difference between the internal volume of the reservoir 1 and the ambient air causes a flow of liquid product contained in the reservoir through the porous pad 9. The porous pad 9 creates a pressure drop that regulates this flow.

The liquid next reaches the microporous membrane 10, which it passes through freely in the absence of foam. The liquid is next discharged through the end piece 7, for example via a channel 17 with a small cross-section, for example a capillary channel) formed in the end piece 7 and allowing the formation of regular drips 18.

When a user releases the pressure that they are exerting on the reservoir 1, their finger 16 ceases to sealingly close off the hole 15. The air is thus free to enter the space formed between the wall 2 and the flexible bag 3. At the same time, the hydrophilic membrane 10 opposes the resuction of air into the flexible bag via the end piece 7. The result is that the volume of liquid product expelled will be completely compensated for by the entry of a corresponding volume of air into the reservoir 1 between the wall 2 and the flexible bag 3.

The dispenser thus allows the packaging of several doses and the delivery of a preservative-free sterile liquid product, in particular of a preservative-free ophthalmic product, containing a surfactant that could interfere with the operation of a bi-functional hydrophilic/hydrophobic sterilising (antibacterial) membrane.

Numerous other aspects that can be implemented on such a dispenser are illustrated in the following figures.

In particular, in order to guarantee the absence of suction of air into the flexible bag 3 and more generally in contact with the liquid product upstream of the hydrophilic membrane 10, various means can be employed as alternatives or in addition to each other. Firstly, a removable cap 19 closing off the end piece 7 can be used. The cap 19 can in particular be adapted to be screwed around the neck 5 of the dispenser. The cap provides impermeability to air by virtue of its method of securing to the neck 5 of the dispenser, and/or by virtue of supplementary means. In particular, a cylinder 20 receiving the end piece 7 can be formed in the cap 19, as well as a stud 21 closing off the channel 17. Thus, once the dispenser is closed, between two product dispensings, no ingress of air via the dispensing end piece 7 can occur.

In the embodiment in FIG. 3, the porous pad 9 is furthermore sized so as to be in contact with or in proximity to the membrane 10. The pad thus forms, between two dispensings of product, a reserve of liquid that persists in wetting the membrane, thus reinforcing its selectivity properties, namely it opposes the passage of gases while allowing passage of liquids. This greatly limits the possibility for air to penetrate under the membrane, upstream thereof.

FIG. 4 presents another embodiment of the invention, wherein, in addition to the use of a cap 19, it is ensured that the membrane remains wetted between two dispensings of product by keeping a very small quantity of liquid downstream of the membrane. This can be achieved by providing the dispenser with a valve 22 in the end piece 7, for example in the immediate vicinity of the end thereof. The valve shown in FIG. 4 is a ball valve, but any other valve technology can obviously be suitable, provided that the valve makes it possible, in particular in addition to the capillarity effect existing in the channel 17, to make the wetting of the membrane 10 by liquid located immediately downstream thereof persist. It is therefore remarkable that it is not necessarily a case of creating a perfect seal downstream of the membrane, but ensuring that the membrane continues to play the role of nonreturn of air that it fulfils through its selectively hydrophilic character.

In the example embodiment shown in FIGS. 1 and 2, the air-inlet means 14 is formed by a hole 15 that can easily be obstructed by the user. The positioning of the hole 15, though it must be natural for the user, can however be adapted according to the dispenser. It can be at the top part of the wall 2, in its middle (in the general direction of extension of the dispenser), or on the bottom 23 of the dispenser.

As illustrated in FIG. 3 and in FIG. 4, the hole 15 can be replaced by a unidirectional valve 24, allowing air to enter the space between the wall 2 and the flexible bag 3 but preventing it from emerging from this space again. The unidirectional valve 24 can be a valve with flexible lips, ball valve, etc. It can be located on the wall 2 as shown in FIG. 4 or on the bottom 23 of the dispenser as shown in FIG. 3. In the embodiment in FIG. 3, the unidirectional valve is included in a hollow 25 formed in the bottom 23 of the dispenser.

Finally, FIG. 5 presents an embodiment in which the air-inlet means 14 is incorporated in the top part of the dispenser, namely, in the example shown, in its neck 5. A conduit 26 guides the incoming air through the air-inlet means towards the space located between the wall 2 and the flexible bag 3. A unidirectional valve 24, forming an air-inlet means 14, is disposed at the mouth of the conduit 26. This embodiment allows discreet incorporation of the air-inlet means and offers a positioning for this means that avoids any risk of unintentional closure during the dispensing of the product contained in the dispenser.

Alternatively to a unidirectional valve, the air-inlet means 14 can be equipped (in any embodiment of the invention) with an element that is permeable to air but is adapted to generate a significant pressure drop (so that the diffusion of air through this element can take place only slowly). A thin cover permeable to air, for example made from silicone, can be used to form this element.

Thus, during the delivery of the product, when the user presses the reservoir 1, a high pressure differential is created but the resistance against expulsion of the product through the end piece 7 is less than the resistance to the passage of air generated by the cover, and the time necessary for dispensing is too short to allow a significant passage of air through the cover.

When the pressure on the reservoir is released, the ingress of air through the end piece 7 being prevented by the hydrophilic membrane 10, the pressure difference between the interior volume of the reservoir and the exterior causes the gradual passage of air through the cover, until the pressures are balanced. The compensation for the volume of product dispensed by the air between the wall 2 and a flexible bag 3 is thus achieved.

FIG. 6 presents another embodiment of the invention, wherein the flexible bag 3 is an attached flexible bag and the air-inlet means includes an element forming a unidirectional valve 24 at the interface between the flexible bag and the element of the dispenser to which the flexible bank is connected.

In the example shown here the flexible bag 3 is connected to the insert 6 (this configuration being moreover applicable to any embodiment including an attached bag).

The element forming a unidirectional valve 24 is open and allows passage of air in the absence of a pressure difference between the exterior of the dispenser and the internal volume of the reservoir, or is configured to open the soon as a small negative pressure is generated in the internal volume of the reservoir. On the other hand, as soon as the internal volume is under overpressure compared with the pressure external to the dispenser (atmospheric pressure), the element forming a unidirectional valve 24 closes and prevents the air from escaping from the space located between the wall 2 and the flexible bag 3.

In particular, the element forming a unidirectional valve 24 can be formed by a thin collar or a flat seal that can be either pressed on the outlet of a conduit 26 connecting the exterior of the dispenser and the internal volume of the reservoir, which closes off the conduit 26, or on the contrary be detached or deformed to leave clear the conduit 26, according to the pressure differential between the interior and the exterior of the dispenser. A cover permeable to air as described above can also be used in a variant of this embodiment.

The embodiment in FIG. 6 offers the advantages of the embodiment in FIG. 5, and is simple to implement.

In all the embodiments presented below, and more generally in all the embodiments of the invention, there can be an advantage in conferring an antibacterial effect on the surfaces of the dispenser located downstream of the membrane 10 and able to be wetted by the liquid product contained in the dispenser. This can be obtained by a treatment of these surfaces or a treatment of the material forming these surfaces. Such a treatment, based on silver ions or zinc ions for example, makes it possible to avoid any bacterial proliferation on these surfaces. If a small quantity of liquid is retained downstream of the membrane 10, the treatment also avoids any bacterial proliferation in this small quantity of liquid.

The antibacterial treatment thus relates, in the example shown, to the internal surfaces of the end piece 7, in particular the channel 17, and where applicable the valve 22. Advantageously, the downstream surface of the membrane 10 is also treated to avoid any bacterial development.

Quite obviously, the various aspects presented above with reference to the figures given by way of example can be combined to form other embodiments of the invention. The dispenser thus developed allows “multidose” packaging of a preservative-free sterile liquid product. Furthermore, the absence of a hydrophobic part on the hydrophilic membrane allows “multidose” packaging of a preservative-free sterile liquid product containing a surfactant. Furthermore, the dispenser takes advantage of the selective character of the hydrophilic membrane with regard to liquids (which it allows to pass) and gases (the passage of which it opposes) to prevent the suction of air into the volume receiving the product. Furthermore, the volume of the bag being adapted to that of the liquid product, all the product can be dispensed, without difficulty, including the last doses present in the dispenser, without residual deformation of the wall of the reservoir.

What is claimed is:

1. A dispenser for drip-dispensing a sterile liquid product containing at least one surfactant, including
a reservoir including a wall defining an internal volume and adapted to deform under the effect of a pressure exerted on the reservoir by a user of the dispenser and to spontaneously resume its original shape after said pressure is released;
the reservoir furthermore including a flexible bag in the internal volume defined by the wall and adapted to contain the sterile liquid product;
an air-inlet means between the wall of the reservoir and the flexible bag;
a head attached to a neck of the reservoir and including a drip end piece including a dispensing opening;
the dispenser furthermore including a hydrophilic sterilising microporous membrane, disposed so as to have the liquid coming from the flexible bag passing through it with a view to delivery of said liquid through the drip end piece;
wherein the dispenser comprises a device configured to keep the membrane wetted by liquid product coming from the reservoir between two dispensings of liquid product;
said microporous membrane being selectively permeable to liquid such that the microporous membrane enables an aqueous liquid to pass while it opposes the passage of gases when it is wetted, and further the microporous membrane opposes the return of air into the flexible bag of the reservoir when the dispenser resumes its original shape after the sterile liquid product is dispensed, the volume of liquid dispensed being compensated for by the entry of a corresponding volume of air between the wall of the reservoir and the flexible bag via the air-inlet means.

2. The dispenser according to claim 1, wherein the flexible bag is formed by delamination of an internal layer of the reservoir.

3. The dispenser according to claim 1, wherein the flexible bag is a retractable flexible bag.

4. The dispenser according to claim 1, wherein the air-inlet means is formed by a hole in the wall of the reservoir adapted to be closed off by a finger of the user.

5. The dispenser according to claim 1, wherein the air-inlet means includes a unidirectional valve, configured to allow air to enter between the wall of the reservoir and the flexible bag and to prevent the air present between the wall of the reservoir and the flexible bag from emerging towards the outside of the reservoir.

6. The dispenser according to claim 1, wherein the device configured to keep the membrane wetted between two instillations is a removable cap, attached to the dispensing head.

7. The dispenser according to claim 1, wherein the device configured to keep the membrane wetted between two instillations includes a valve positioned between the membrane and the dispensing opening of the drip end piece.

8. The dispenser according to claim 1, wherein the dispenser further includes a porous pad located between the reservoir and the microporous membrane and disposed so as to have the liquid coming from the flexible bag passing through the porous pad with a view to delivery of said liquid through the drip end piece.

9. The dispenser according to claim 1, wherein the microporous membrane has a pore size of less than 0.45 μm and preferably less than or equal to 0.22 μm.

10. The dispenser according to claim 1, wherein the surfaces of the dispenser located between the microporous membrane and the dispensing opening carry a bactericidal coating, for example based on silver ions or zinc ions.

11. The dispenser according to claim 1, wherein the microporous membrane includes a bactericidal agent, for example based on silver ions or zinc ions.

12. The dispenser according to claim 1, said dispenser including in its reservoir a preservative-free sterile liquid ophthalmic product containing at least one surfactant.

13. The dispenser according to claim 12, wherein the sterile liquid ophthalmic product includes between 0.1% and 5% surfactant by volume.

14. The dispenser according to claim 12, wherein the at least one surfactant includes one or more of the following agents:
Polyethylene glycol 400 (PEG 400);
Polyoxyethylene sorbitan monooleate (polysorbate 80);
Polyoxyethylene sorbitan monolaurate (polysorbate 20);
Ethylene oxide, propylene oxide;
Methyloxirane polymer;
Castor oil PEG-35;
Hydrogenated castor oil PEG-40;
Macrogol cetostearyl ether;
Polyoxypropyl glycol 407;
Vitamin E (polyethylene glycol succinate);
Macrogol hydroxystearate 15;
Caprylocaproyl macrogol-8 glycerides;
2-(2-ethoxyethoxy) ethanol;
Polyvinylpyrrolidone.
